(19) United States
(12) Patent Application Publication
(10) Pub. No.: US 2012/0097165 A1
(43) Pub. Date: Apr. 26, 2012

(54) OXYGEN MASK WHICH IS CONNECTED EXTERNALLY TO A CARBON DIOXIDE DENSITY TESTER AND FACILITATES A PATIENT TO BREATHE IN OXYGEN

(76) Inventor: PO-YU KUO, Panchiao Taipei (TW)

(21) Appl. No.: 12/910,530

(22) Filed: Oct. 22, 2010

(51) Int. Cl. A61M 16/00; A61B 5/08

(52) U.S. Cl. 128/205.13; 600/532

(57) ABSTRACT

The present invention discloses an oxygen mask which is connected externally to a carbon dioxide density tester and facilitates a patient to breathe in oxygen. The oxygen mask comprises a mask and a tube unit, and only covers a nose of a patient without covering a mouth. An opening of a respiration tube of the tube unit faces upward to facilitate connecting externally with a resuscitation bag. The respiration tube is provided with a carbon dioxide discharge tube which is integrally formed with the respiration tube and can be externally connected with a carbon dioxide density tester, allowing a paramedic to observe density of carbon dioxide exhaled from the patient, without resulting in a leak to the oxygen mask, which affects the patient to breathe in oxygen.

### BACKGROUND OF THE INVENTION

a) Field of the Invention

The present invention relates to an oxygen mask which is connected externally to a carbon dioxide density tester and facilitates a patient to breathe in oxygen and more particularly to an oxygen mask that can be connected externally to a carbon dioxide density tester, allowing the oxygen mask to have the dual function of supplying oxygen and testing density of carbon dioxide exhaled from the patient.

b) Description of the Prior Art

In a hospital without sufficient equipment to test density of carbon dioxide exhaled by a patent, doctors will usually employ a visual test. Therefore, all the oxygen masks in the hospital are required to be made of a transparent PVC material. When the patient exhales carbon dioxide, mist will remain in the oxygen mask, allowing the doctors to determine whether the patient is breathing during treatment. However, an accuracy of this visual test cannot be assured and the doctors will also expect equipment readings for determination.

For a hospital with the equipment, as there are no proper oxygen masks to be used by paramedics, when doctors are testing density of carbon dioxide exhaled from a patent, referring to FIG. 1, an end of an oxygen mask 100 will be normally inserted with a guide tube 200. An end of the guide tube 200 is connected to a carbon dioxide tester (not shown in the drawing), and the other end passes through directly the oxygen mask 100 to touch the patient's skin. This condition will result in a gap 101 on the oxygen mask 100, allowing that the oxygen mask 100 cannot be tightly engaged with the patient's face and oxygen can escape from the gap 101 at an edge of the guide tube 200 to affect the patient to breathe in oxygen.

### SUMMARY OF THE INVENTION

Accordingly, the primary object of the present invention is to provide an oxygen mask which is connected externally to a carbon dioxide density tester and facilitates a patient to breathe in oxygen, wherein an ordinary oxygen mask is provided with a carbon dioxide discharge tube which can be connected externally to a carbon dioxide density tester. Hence, in addition to that the oxygen mask itself can be connected externally to a resuscitation bag to supply the patient with oxygen, density of carbon dioxide exhaled from the patient can be tested by a paramedic very conveniently.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a schematic view illustrating actions of a prior art.

FIG. 2 shows a schematic view of the present invention.

FIG. 3 shows an exploded view of the present invention.

FIG. 3-1 shows a bottom schematic view of a tube unit of the present invention.

FIG. 3-2 shows a partial cross-sectional view of the tube unit of the present invention.

FIG. 4 shows a schematic view illustrating actions of the present invention.

FIG. 5 shows another schematic view illustrating actions of the present invention.

### DETAILED DESCRIPTION OF THE PREFERRED EMBODIMENTS

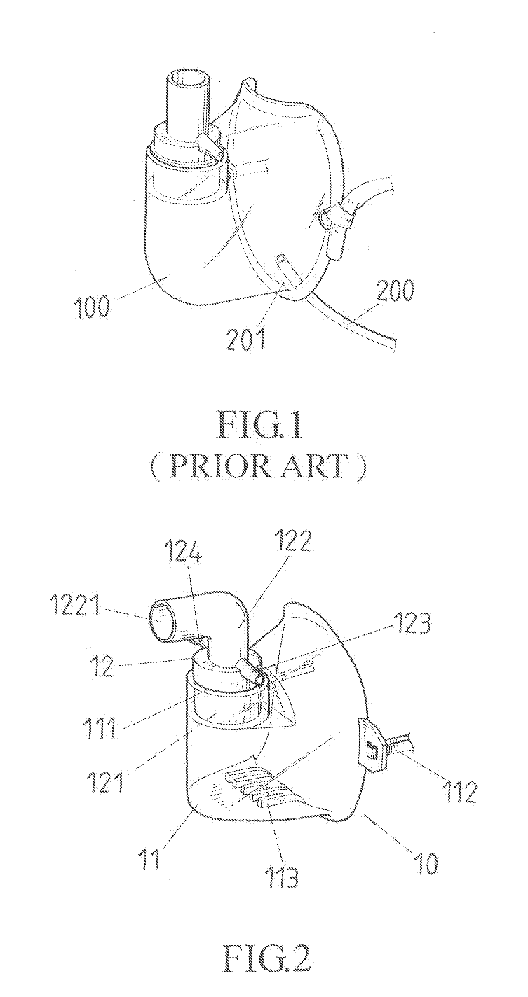

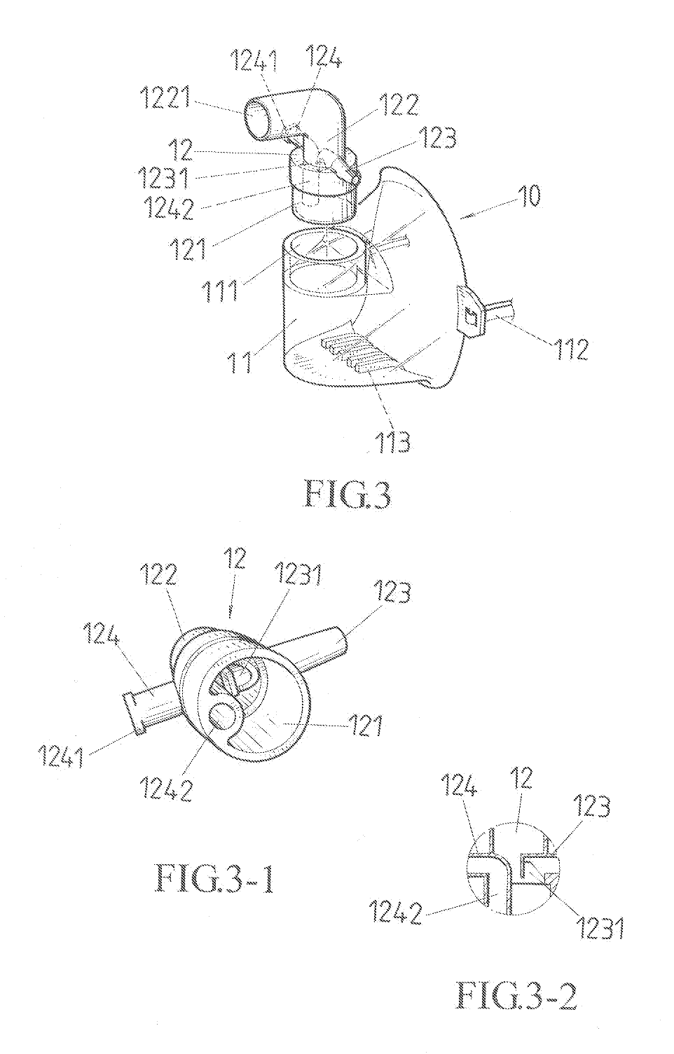

Referring to FIGS. 2 and 3, an oxygen mask 10 of the present invention comprises a mask 11 and a tube unit 12, openings at two sides of the mask 10 are tied with straps 112 to sheath the mask 10 at a patient's head and a top part at a front side is provided with a latch slot 111 to latch the tube unit 12.

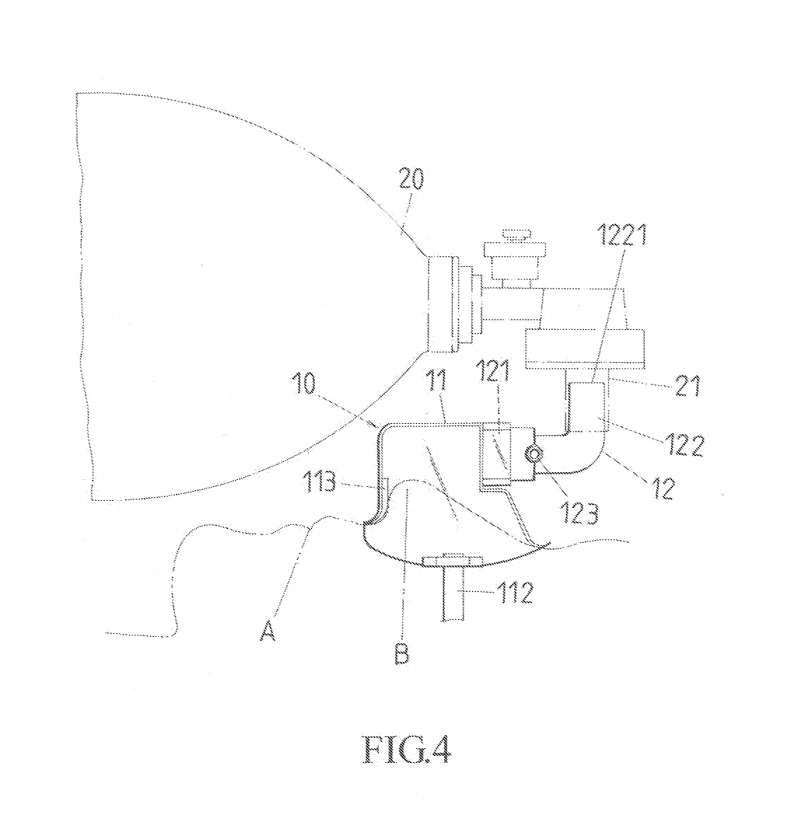

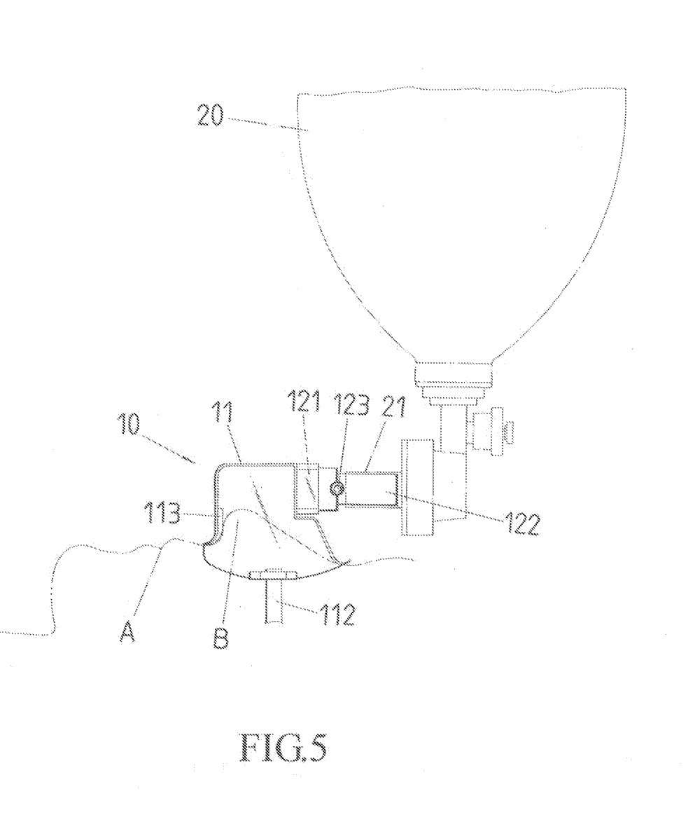

An inner bottom of the mask 11 of the oxygen mask 10 is provided with plural pillars 113, which form space into an irregular groove, preventing that a paramedic puts the mask 11 close to a nostril end B of the patient carelessly (as shown in FIG. 4), so as to assure that the patient can breathe smoothly and avoid an accident.

An opening 1221 of a respiration tube 122 of the tube unit 12 faces upward and is in an L-shape to facilitate connecting externally with a resuscitation bag 20 (as shown in FIG. 4). A transversal air inlet tube 123 is provided at a center of an exterior side of the tube unit 12 and is extended to an inner tube 1231 inside the tube unit 12 to be bended in an L-shape (as shown in FIG. 3-2).

Referring to FIG. 3-1, another end of the respiration tube 122 of the tube unit 12 is provided with a carbon dioxide discharge tube 124 which is integrally formed with the respiration tube 122. An opening of the carbon dioxide discharge tube 124 is provided with a tenon 1241 and when the carbon dioxide discharge tube 124 is connected externally to a carbon dioxide density tester, the tenon 1241 can be latched after rotation (not shown in the drawings). Referring to FIG. 3-2, an inner tube 1242 of the carbon dioxide discharge tube 124 extending into the tube unit 12 is in an L-shape.

When the patient breathes in air from the air inlet tube 123 of the tube unit 12, carbon dioxide will be exhaled from the opening 1221 of the respiration tube 122 when he or she breathes out; whereas, part of carbon dioxide exhaled from the patient will be expelled from the carbon dioxide discharge tube 124 to facilitate testing density of carbon dioxide exhaled from the patient. Through a repeated test by a hospital, this oxygen inhaling method is the best way to facilitate the patient breathing in oxygen and can supply more oxygen to breathe in than an ordinary respiration tube.

Referring to FIG. 4, when the patient lies down flat, the oxygen mask 10 can cover the nose B without coving a mouth A, and the opening 1221 of the L-shaped respiration tube 122 of the tube unit 12 faces upward to facilitate the paramedic to connect a connection tube 21 at a front end of the resuscitation bag 20.

Referring to FIG. 5, when the patient lies down flat, the oxygen mask 10 can cover the nose B without coving the mouth A, and the respiration tube 122 above the tube unit 12 is horizontally connected to the connection tube 21 of the resuscitation bag 20.

Concluding from above, the present invention provides an oxygen mask which is connected externally to a carbon dioxide density tester and facilitates a patient to breathe in oxygen, wherein a respiration tube of an ordinary mask for the patient to breathe in oxygen is provided with a carbon dioxide discharge tube which is integrally formed with the respiration tube and can be connected externally with a carbon dioxide density tester. Therefore, directly from the carbon dioxide density tester, a paramedic is able to clearly determine density of carbon dioxide which is exhaled from the patent and this added apparatus will not affect the function of taking oxygen of the oxygen mask itself.

What is claim is:

1. An oxygen mask which is connected externally to a carbon dioxide density tester and facilitates a patient to breathe in oxygen, wherein the mask covers a nose without covering a mouth and a top part at a front side of the mask is provided with a latch slot to latch a tube unit; an opening of a respiration tube of the tube unit facing upward and being in an L-shape to facilitate connecting externally with a resuscitation bag, an air inlet tube being located at a center of an exterior side of the tube unit, an inner tube of the air inlet tube being extended into the tube unit to be bended in an L-shape and an inner bottom of the mask being provided with plural pillars which form space into an irregular groove; the primary characteristic being that the respiration tube of the tube unit is provided with a carbon dioxide discharge tube which is connected externally with a carbon dioxide density tester, with that an inner tube of the carbon dioxide discharge tube extending into the tube unit is in an L-shape.

2. The oxygen mask which is connected externally to a carbon dioxide density tester and facilitates a patient to breathe in oxygen, according to claim 1, wherein the carbon dioxide discharge tube is integrally formed with the respiration tube.

3. The oxygen mask which is connected externally to a carbon dioxide density tester and facilitates a patient to breathe in oxygen, according to claim 1, wherein an opening of the carbon dioxide discharge tube is provided with a tenon to latch with the carbon dioxide density tester.
